(19) United States
(12) Patent Application Publication
(10) Pub. No.: US 2002/0133060 A1
(43) Pub. Date: Sep. 19, 2002

(54) Combined nasal speculum and aspirator

(76) Inventor: Donald E. Doyle, Pascagoula, MS (US)

Correspondence Address: Roger A. Marrs, Suite 1220, 15233 Vetura Blvd., Sherman oaks, CA, 91403, US

(21) Appl. No.: 10/096,622

(22) Filed: Mar. 12, 2002

Related U.S. Application Data
(60) Provisional application No. 60/275,123, filed on Mar. 13, 2001.

(51) Int. Cl. A61B 1/32

(52) U.S. Cl. 600/210

(57) ABSTRACT

A pair of tongs are joined together by a pivot wherein one end of each of the respective tongs includes a handle portion and a spring biasing mechanism which expands against the respective handle portions so that the opposite end of the tongs carrying cantilevered prongs are normally biased together. The prongs curve at a 90° angle and extend outwardly from the instrument at the distal end in curvatures. A selected one of the prongs is provided with an elongated slot for mounting of a suction tube. One end of the suction tube terminates adjacent the curved tip while the opposite end of the suction tube is connected to a source of suction.

Priority Based on Ser. No. 60-275,123 filed Mar. 13, 2001.

### BACKGROUND OF THE INVENTION

1. Field of the Invention

The present invention relates to the field of nasal instruments employed in otorhinolaryngological surgical procedures and, more particularly, to a novel combined nasal speculum with a built-in aspirator allowing surgery to be performed while providing simultaneous suctional removal of blood and fluids.

2. Brief Description of the Prior Art

During the performance of nasal surgery, several separate instruments are required to be inserted through a very narrow nasal opening. Not only is the operating and visual area limited, but such portions of the anatomy as the maxillary crest and the vomer are a source of blood which can profusely bleed. It is for the bleeding from these structures that one of the inserted instruments is an aspirator for removing blood by suctional techniques. Another inserted instrument is a nasal speculum having a pair of manually spreadable prongs that expand the nasal passageway to increase the visual and operating area.

Difficulties and problems have been encountered in performing nasal surgery which stem from the need to insert several instruments into a narrow nasal passageway in order to provide a surgeon with a satisfactory view and operating area to continue the surgery. It must be noted that there are many instances where the septum has very irregular bone or cartilage due to injury or to congenital causes that require very delicate dissection in order to preserve the integrity of the membrane. Conventional speculae are of varying lengths and taper downwardly so that the leading edge is about 50% the width of the part closest to the hand. Therefore, the instrument occupies a substantial area of the nasal passageway which reduces the available area or space in which to insert and manipulate surgical instruments. The volume of tissue displacement is reduced to further complicate the surgical procedure.

The surgeon employs instruments such as chisels or hard strong bending tools to remove parts of the maxillary crest and/or vomer. Bleeding usually occurs in this area requiring cauterization. The surgeon inserts a speculae with one hand and a dissecting tool in the other hand. When encountering bleeding of significance, the dissecting tool must be withdrawn and put aside while a separate suction device or aspirator is then placed into the nasal passageway to clear the operative field. Often, as the suction device has been used to clear the field and removed from the nasal passageway, the blood again accumulates making the operative procedure very tedious and prolonged.

Therefore, a long-standing need has existed to provide a single instrument capable of performing multiple functions for insertion into a nasal passageway or cavity having limited access and operating space. The single or unitary instrument should at least maintain the nasal passageway open as much as possible while simultaneously providing suction for blood and mucous removal. The surgeon need only to operate a single instrument with one hand to accommodate multiple functions while the other hand is free to manipulate other surgical instruments.

### SUMMARY OF THE INVENTION

Accordingly, the above problems and difficulties are avoided by the present invention which provides a novel combined nasal speculum and aspirator which includes a pair of tongs that are joined together by a pivot. One end of each of the respective tongs includes a handle portion. A spring biasing mechanism is illustrated as expanding against the inside or opposing surfaces of the respective handle portions so that the opposite end of the tongs are normally biased together. This opposite end of the tongs includes arms which terminate in prongs that curve at a 90° angle and cantilever outwardly from the instrument at the distal end in curvatures. It is to be particularly noted that a selected one of the prongs may be provided with an elongated slot for some applications for accommodating passage of a suction tube. One end of the suction tube terminates adjacent the curved tip while the opposite end of the suction tube is connected to a source of suction. The suction tube is on the inside of prong ahead of the slot while the tube or hose is on the outside of the instrument and traverses on the outside of the instrument along the prong.

Therefore, it is among the primary objects of the present invention to provide a novel combined nasal speculum and aspirator which is specifically designed for nasal and/or nasal septal surgery and further which incorporates a means to remove blood from the surgical field so as to allow the surgeon to continue operating without the necessity of discontinuing his surgery to pick up additional, separate suction devices or to have an assistant place a separate suctioning device in the operating field thereby interrupting the actual dissection.

Another object of the present invention is to provide a novel combined nasal speculum and aspirator which provides suction within a nasal speculum which not only increases the area of the operating field but can control bleeding from adjacent structures as an operation is being performed.

Still another object of the present invention is to provide a novel medical instrument in a single unitary construction which will not only expand nasal passageways to provide an increased field of operation but which will provide suction or aspiration of blood from the expanded field.

Yet another object of the present invention is to provide a single instrument for expanding the nasal passageway and for aspirating blood from the field which can be operated by one hand of a surgeon during a medical procedure.

### BRIEF DESCRIPTION OF THE DRAWINGS

The features of the present invention which are believed to be novel are set forth with particularity in the appended claims. The present invention, both as to its organization and manner of operation, together with further objects and advantages thereof, may best be understood with reference to the following description, taken in connection with the accompanying drawings in which:

FIG. 1 is a rear perspective view showing the novel combined nasal speculum and aspirator incorporating the present invention;

FIG. 2 is a front elevational view of the invention shown in FIG. 1 as taken in the direction of arrow 2-2 thereof;

FIG. 3 is a top plan view of the inventive instrument shown in FIG. 1;

FIG. 4 is a rear perspective view of another version of the surgical instrument incorporating the present invention;

FIG. 5 is a transverse cross-sectional view of the surgical instrument shown in FIG. 4 as taken in the direction of arrows 5-5 thereof; and

FIG. 6 is a transverse cross-sectional view taken in the direction of arrow 6-6 of FIG. 4.

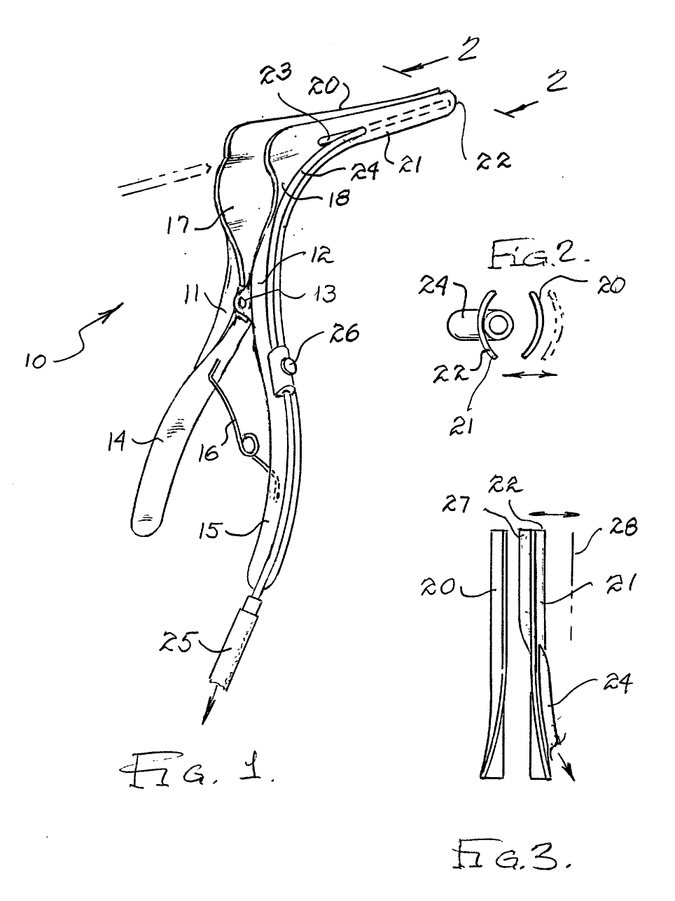

### DESCRIPTION OF PREFERRED EMBODIMENT

Accordingly, the above problems and difficulties are avoided by the present invention which provides a novel combined nasal speculum and aspirator which is illustrated in the general direction of arrow 10 that includes a pair of tongs 11 and 12 that are joined together by a pivot 13. One end of each of the respective tongs includes a handle portion 14 and handle portion 15. A spring biasing mechanism 16 is illustrated as expanding against the inside or opposing surfaces of the respective handle portions so that the opposite end of the tongs 11 and 12 are normally biased together. This opposite end of the tongs includes arms 17 and 18 which terminate in prongs 20 and 21 that curve at a 90° angle and lever outwardly from the instrument 10 at the distal end in curvatures such as curved tip 22. It is to be particularly noted that a selected one of the prongs 20 and 21 is provided with an elongated slot 23 for accommodating passage of a suction tube 24. One end of the suction tube terminates adjacent the curved tip 22 while the opposite end of the suction tube is connected to a source of suction, such as a pump or the like and a connection to such a source is indicated by tube 25. The suction tube 24 is on the inside of prong 21 ahead of the slot 23 while the tube or hose 24 is on the outside of the instrument and traverses on the outside of the instrument along prong 12. Suitable means are employed for retaining the hose in position and a control device 26 is employed for controlling the amount of suction supplied to the tip of the hose adjacent to the curved tip 22 of prong 21.

Referring now in detail to FIG. 2, it can be seen that the prongs 20 and 21 are normally biased in the position shown in solid lines by the spring 16. However, when the handle portions 15 and 16 are pulled together against the biased of spring 16, the distance between the opposing surfaces of the prongs 20 and 21 are expanded to the position shown in broken lines. However, the suction hose 24 resides along the inside surface of prong 21 and the terminating end of the hose is in position to aspirate the operational area when the prongs 20 and 21 are inserted into the nasal passageway and expanded.

Referring now in detail to FIG. 3, it can be seen that the prongs 20 and 21 are relatively close to one another as forcibly urged by the spring 16. However, when the prongs are pivoted so that the prongs are expanded to the position indicated in broken lines by numeral 28, the passageway to be operated upon has been enlarged and other physical instruments have more available space in which to be manipulated. Simultaneously, the end 27 of the suction tube 24 is in position to aspirate or remove any blood, fluid, or other debris from the operating area.

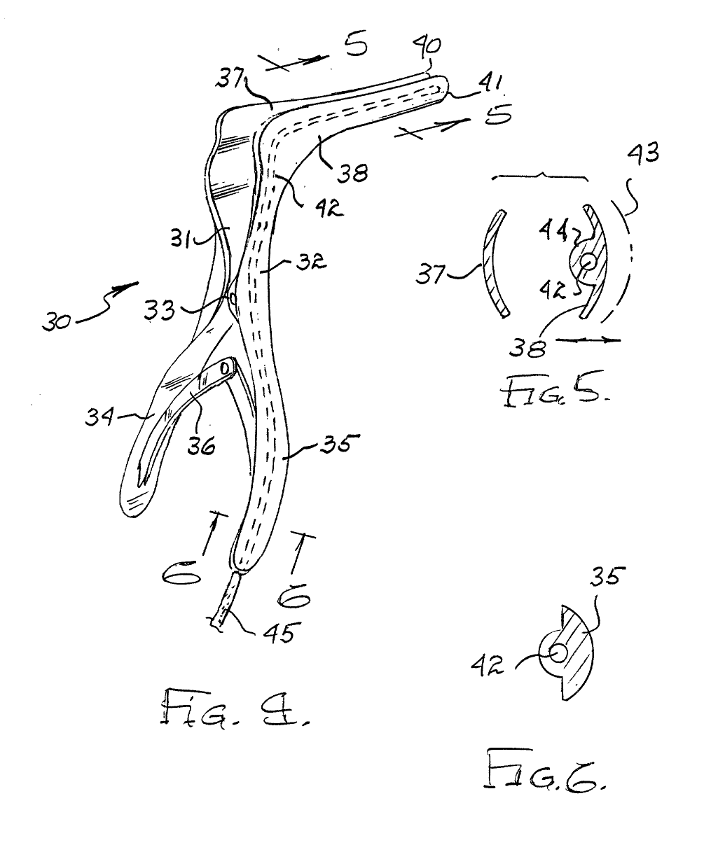

Referring now in detail to FIGS. 4-6, another version of the novel combined nasal speculum and aspirator is illustrated in the general direction of arrow 30 which includes tongs 31 and 32 connected together by a pivot 33 in a scissors-like fashion. The lower end of each of the tongs 31 and 32 include hand-grips 34 and 35 which are normally biased away from one another by means of a spring biasing means 36. The opposite end of the respective tongs terminate in an outwardly projecting pair of prongs 37 and 38 which cantilever outwardly in a lateral direction from the longitudinal axis of the tongs 31 and 32. The respective prongs terminate into ends 40 and 41 in a similar fashion to the previously described instrument. Suction means are provided which are integrally formed in a selected one of the prongs. In the illustration, an internal duct or passageway is identified by numeral 42 and extends from one end of the handle portion 35 up through the mid-section of the tong 32 and through the prong 38 to terminate in an opening adjacent to the curved tip 41.

As illustrated in FIG. 5, the prongs 37 and 38 are indicated in relative close, spaced-apart relationship due to the spring bias of the spring means 36. However, when the handle portions 34 and 35 are compressed against the spring means 36, the prongs expand to the position shown in broken line and identified by numeral 43. The continuous passageway 42 is illustrated as integrally formed with the prong 38 and is internal of a curved sidewall 44.

FIG. 6 illustrates that the passageway 42 is conducted through the prong 32 and is also carried in the handle portion 35 of the latter prong. A suction tube 45 can be connected to the passageway 42 by any conventional means and the opposite ends is connected to a suction pump for creating sufficient suction at end 42 to withdraw blood, mucous or other foreign matter present in the operating zone of the nasal passageway.

In view of the foregoing, it can be seen that the speculum may be of varying lengths from 2 cm. to 8 cm., with a distal blade width not less than 60% of the width of the base blade of the speculum and no more than 90°. By having the blades tapered, it is easier to introduce the prongs or blades into a narrow opening and easier to extract. Also, it is preferred that the external surface of the instrument be “ebonized” since most surgeons employ lighting to see the operating field and if the instrument is shiny, the light reflects back into the surgeons eyes making it painful to operate. The instrument of the present invention is made specifically for nasal and/or nasal septal surgery and incorporates suction means to simultaneously remove blood from the operating field while the surgeon continues the operation with additional instruments and, therefore, operates without the necessity of discontinuing his surgery to pick up a separate suction device or have an assistant place a separate suction device in the operating field which would interrupt the actual dissection.

While particular embodiments of the present invention have been shown and described, it will be obvious to those skilled in the art that changes and modifications may be made without departing from this invention in its broader aspects and, therefore, the aim in the appended claims is to cover all such changes and modifications as fall within the true spirit and scope of this invention.

What is claimed is:

1. A combined nasal speculum and aspirator device comprising:
a pair of tongs having a mid-section of each tong joined by a pivot;
each tong having a handle section and an arm section separated by said mid-section;
each arm section terminating in an outwardly projecting and cantilevered prong; and
an elongated conduit disposed on a selected one of said prongs and one end of said conduit terminates in an open intake port and another end of said conduit operably coupled to a source of suction.

2. The device defined in claim 1 wherein:
each prong carried on each tong curved from said arm at an angle within a range of 70°-110°.

3. The device defined in claim 2 wherein:
each of said prongs terminates in a rounded tip.

4. The device defined in claim 3 wherein:
each of said prongs includes a curved cross-section defining a curved surface, wherein said curved surfaces of said prongs oppose each other.

5. The device defined in claim 4 wherein:
said conduit is a length of hose; and
a selected one of said prongs having a slot conducting said one end of said conduit from a fixed position on the outside of said prong to a fixed position on the inside of the prong.

6. The device defined in claim 4 wherein:
said conduit is integrally formed with said tong and said prong to provide a unitary construction; and
said conduit disposed along the full length of said tong and said prong.

7. The device defined in claim 6 including:
expansion means operably disposed between said pair of tongs normally urging said pair of prongs together.

8. A nasal speculum and aspiratory combined in a single unitary construction comprising:
a pair of elongated tongs joined at their mid-section by a pivot to permit articulation thereof;
said tongs having a handle section at one end and an outwardly cantilevered prong terminating in a curved tip;
a selected one of said tongs having an internal open ended conduit with one open end thereof at said handle section and another open end at said curved tip;
a source of suction coupled to said open end at said handle section; and
spring biasing means carried between said handle sections yieldably urging said prong together.

9. The device defined in claim 8 wherein:
said prongs are curved in cross section with inside surfaces facing each other; and
said conduit resides along side inside surface of a selected one of said prongs.

10. The device defined in claim 9 wherein:
said prong carried on each of said tongs extends outwardly therefrom at an angle within a range of 70°-110°.

11. The device defined in claim 10 wherein:
said spring biasing means is an expansion spring yieldably urging said handle section apart.
